(19) United States
(12) Patent Application Publication
(10) Pub. No.: US 2007/0137462 A1
(43) Pub. Date: Jun. 21, 2007

(54) Wireless communications device with audio-visual effect generator

(75) Inventors: Mark A. Barros, Wellington, FL (US); Von A. Mock, Boynton Beach, FL (US); Charles P. Schultz, North Miami Beach, FL (US)

Correspondence Address: FLEIT, KAIN, GIBBONS, GUTMAN, BONGINI;& BIANCO P.L., 551 N.W. 77TH STREET, SUITE 111, BOCA RATON, FL, 33487, US

(73) Assignee: MOTOROLA, INC., SCHAUMBURG, IL (US)

(21) Appl. No.: 11/305,371

(22) Filed: Dec. 16, 2005

(51) Int. Cl. A61B 7/00; G10D 9/00; G06K 9/00; A61B 5/02; A61B 5/103

(52) U.S. Cl. 84/453; 600/508; 600/481; 600/586; 600/595; 600/587; 382/124

(57) ABSTRACT

A wireless personal communications device (106) that has a wireless communications circuit (704) that communicates over a cellular communications system and a user input sensor (306, 404, 410, 412, 502) that detects an input from a user (104, 108). An included audio-visual effect generator (210) generates an audio-visual effect based upon input detected by the user input sensor (306, 404, 410, 412, 502). A collaborative audio-visual effect creation system (110) accepts audio visual effects from multiple sources (106), uses a multiple user wireless data communications system to wirelessly communicate data among multiple wireless personal communications devices (106), accepts rating information from the wireless personal communications devices (106) and produces an audio-visual output that is derived received audio-visual effects based upon the rating information.

### FIELD OF THE INVENTION

The present invention generally relates to the field of audio-visual effects generators and more specifically to wireless personal communications devices that generate and mix audio-visual effects to be communicated over wireless data links.

### BACKGROUND OF THE INVENTION

“Air guitaring” or “Air drumming” are terms used to describe the act of strumming an invisible guitar in the air or pounding an invisible drum in unison with the music being played. Air guitaring and air drumming are usually performed by people who are listening to music, but these are purely physical acts that in no way affects the music being played. Air guitaring and air drumming do provide an indescribable level of pleasure to the user as is evidenced by the fact that so many people do it.

Professional and casual musicians devote time and money to their craft. A good portion of this money is spent on equipment for instrument tuning, effects, and accompanying devices such as drum machines and practice amps. Additionally, time and money is spent on getting together with other musicians at a location where they are all able to bring and set up their equipment. These musicians are also limited to meeting in areas where there are sufficient resources, such as the size of the area, availability of sufficient electrical power, and acoustics. These areas must also be suitable for playing music such as being located where the noise is not offensive. The effort required by each musician to bring his or her equipment to a location is a disincentive to casual jam sessions or assembling large groups of musicians to either play together or to join together into smaller sub-groups that each takes turns playing for a short time. Further, participants or even the audience in general have no automated method in which to provide feedback to affect which musicians are selected to participate in the currently playing or subsequently playing sub-group.

Therefore a need exists to overcome the problems with the prior art as discussed above.

### SUMMARY OF THE INVENTION

According to an embodiment of the present invention, a wireless personal communications device includes a hand held housing and a wireless personal communications circuit that is mechanically coupled to the housing. The wireless personal communications circuits communicate over a commercial cellular communications system. The wireless personal communications device further includes a user input motion sensor that is mechanically coupled to the housing and that is able to detect at least one motion performed by a user in association with housing. The wireless personal communications device also includes an audio-visual effect generator that is communicatively coupled to the user input motion sensor and that generates an audio-visual effect based upon motion detected by the user input motion sensor.

According to another aspect of the present invention, a collaborative audio-visual effect creation system includes a plurality of audio-visual effect generators that generate a plurality of audio-visual effects. Each respective audio-visual effect generator within the plurality of audio-visual effect generators generates a respective audio-visual effect within the plurality of audio-visual effects. The collaborative audio-visual effect creation system also includes a multiple user wireless data communications system that wirelessly communicates data among a plurality of wireless personal communications devices. The collaborative audio-visual effect creation system further includes a contribution controller that accepts rating information from each wireless personal communications device within the plurality of wireless personal communications devices and produces an audio-visual output derived from a plurality of audio-visual effects based upon the rating information

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates an ad-hoc jam session configuration according to an exemplary embodiment of the present invention.

FIG. 2 illustrates a circuit block diagram for an audio-visual effect generation and mixing apparatus contained within a wireless personal communications device, according to an exemplary embodiment of the present invention.

FIG. 3 illustrates a front-and-side view of an exemplary monolithic wireless personal communications device according to an exemplary embodiment of the present invention.

FIG. 4 illustrates a rear-and-side view of an exemplary monolithic wireless personal communications device according to an exemplary embodiment of the present invention.

FIG. 5 illustrates a cut-away profile of a flip-type cellular phone, according to an exemplary embodiment of the present invention.

FIG. 6 illustrates a collaborative audio-visual effect base station apparatus block diagram according to an exemplary embodiment of the present invention.

FIG. 7 illustrates a wireless personal communications device apparatus block diagram according to an exemplary embodiment of the present invention.

FIG. 8 illustrates a hand waving monitor apparatus as incorporated into the exemplary embodiment of the present invention.

FIG. 9 illustrates a sound effect generation processing flow in accordance with an exemplary embodiment of the present invention.

FIG. 10 illustrates a collaborative audio-visual effects creation system processing flow in accordance with an exemplary embodiment of the present invention.

### DETAILED DESCRIPTION

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The terms “a” or “an”, as used herein, are defined as one or more than one. The term plurality, as used herein, is defined as two or more than two. The term another, as used herein, is defined as at least a second or more. The terms including and/or having, as used herein, are defined as comprising (i.e., open language).

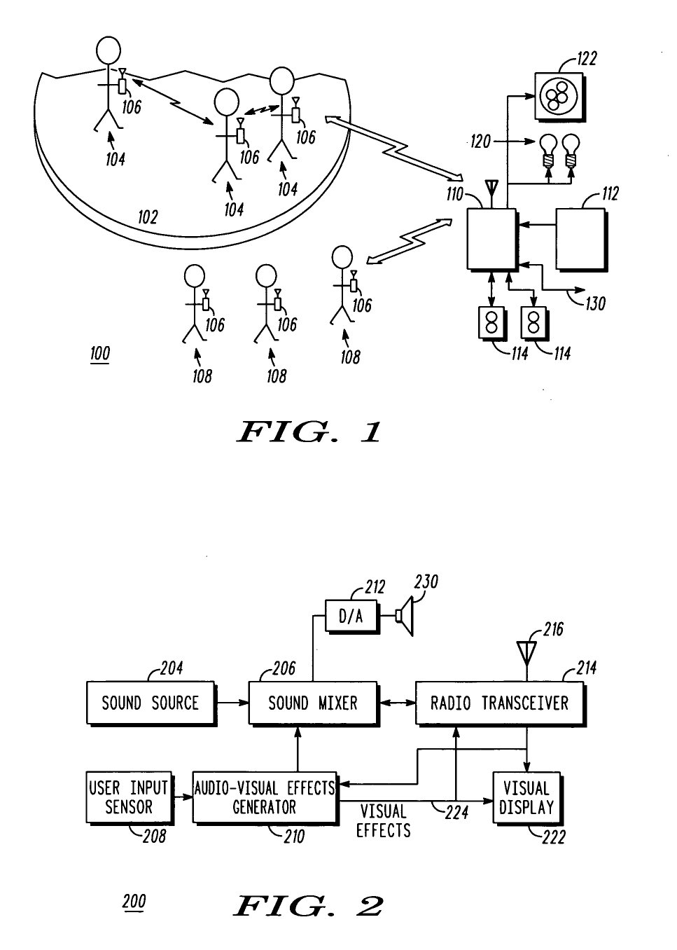

FIG. 1 illustrates an ad-hoc jam session configuration 100 according to an exemplary embodiment of the present invention. The exemplary ad-hoc jam session configuration 100 includes a venue with a stage 102 on which three (3) musicians 104 stand. Each of these three musicians 104 is holding an exemplary wireless personal communications device 106 that further includes additional components, as described in detail below, to allow generation of audio-visual effects. Through the proper use of these exemplary wireless personal communications devices 106, the musicians 104 are able to collaboratively generate audio-visual effects, such as music, that can be played in the venue or communicated to other geographic locations. In addition to use of the wireless personal communications devices 106, musicians 104 are able to use conventional musical instruments which are able to be connected to either a wireless personal communications device 106 or directly to a music mixer or other type of audio-visual effect base station.

The exemplary wireless personal communications devices 106 include data communications circuits that support wireless data communications between and among all of the exemplary wireless personal communications devices 106. The exemplary embodiment includes data communications circuits that conform to the Bluetooth® standard and also include data communications circuits that conform to data communications standards within the IEEE 802.11 series of standards. The IEEE 802.11 standards are available from the Institute of Electrical and Electronic Engineers. The wireless distribution of data among multiple wireless personal communications devices through these data communications standards is known to ordinary practitioners in the relevant arts in light of the present discussion.

Audio-visual effects generated by the wireless personal communications devices 106 held by musicians 104 are able to communicate their generated audio-visual effects among each other over wireless data links that operate as commercial cellular links, ad-hoc Bluetooth groups or peer-to-peer networks. Music mixing circuits within the wireless personal communications devices 106 receive the audio-visual effects transmitted by other wireless personal communications devices 106 and produce a composite audio-visual effect signal that is able to be reproduced by that wireless personal communications device 106 or communicated to another device.

In this example, the musical sound content is produced in digital form by the wireless personal communications devices 106 and that musical sound content is then wirelessly communicated to a central base station 110. In the exemplary embodiment of the present invention, musical sound content is able to include, for example and without limitation, vocally produced content such as speech, singing, and rapping. Central base station 110 of the exemplary embodiment is also able to accept electrical signals representing sound from a sound source 112. Sound source 112 is able to be, for example, a juke box or any storage of recorded music. Sound source 112 can further produce an announcer's message, a singer, or any other sound signal. In this example, the composite sound produced by the central base station 110 is produced through attached speakers 114.

In addition to the musicians 104 in this exemplary configuration, there are a number of spectators 108 in the venue that each has a wireless personal communications device 106. These spectators 108 are able to use their wireless personal communications devices 106 to generate additional audio-visual effects, such as their own sound signals or commands for visual effects. These spectators 108 in the exemplary embodiment are further able to provide feedback, such as votes or quality ratings for each of the musicians 104 or other spectators 108.

The base station 110 of the exemplary embodiment includes a wireless data communications system, described below, that receives data containing the musical signals and other audio-visual effects produced by the wireless personal communications devices 106 held by musicians 104 and to also receive audio-visual effects and voting data generated by wireless personal communications devices 106 held by spectators 108. The wireless data communications system contained within base station 110 is part of a multiple user wireless data communications system that wirelessly communicates data among many wireless personal communications devices 106. The base station 110 produces a composite sound signal that includes one or more channels of sound information based upon the received musical signals and audio-visual effects generated by and received from the wireless personal communications devices 106 held by the musicians 104 and spectators 108.

The composite sound in the exemplary embodiment is reproduced through attached speakers 114 and wirelessly transmitted to each wireless personal communications device 106. The wireless personal communications devices 106 receive a digitized version of the composite audio signal and reproduce the audio signal through a speaker or personal headset that is part of, or attached to, the wireless personal communications device 106. Further embodiments of the present invention do not include attached speakers 114 and only reproduce sound through the speakers or headsets of the wireless personal communications devices 106. The composite audio signal in the exemplary embodiment is also communicated to other locations over a data link 130, such as the Internet. The base station 110 is further able to receive musical signals or other audio-visual effects from remote locations, such as other venues or from individual musicians, over the data link 130. Users in such remote locations are further able to provide feedback, such as votes or quality ratings for the musicians 104 or other spectators 108, over the data link 130. As an example, a remote venue is able to contain another base station 110 that receives signals from wireless personal communications devices 106 that are within that remote venue.

The base station 110 of the exemplary embodiment further controls show lights 120 and a kaleidoscope 122 to present a visual demonstration in the venue. The show lights 120 and kaleidoscope 122 are controlled at least in part by audio-visual effect commands generated by the wireless personal communications devices 106 held by the spectators 108 or musicians 104.

FIG. 2 illustrates a circuit block diagram for an audio-visual effect generation and mixing circuit 200 contained within a wireless personal communications device 106 as shown in FIG. 1, according to an exemplary embodiment of the present invention. The audio-visual effect generation and mixing circuit 200 includes a radio transceiver 214 that performs bi-directional wireless data communications through antenna 216. Radio transceiver 214 transmits, over a wireless data link, sound signals that are encoded in a digital form and that are produced within the audio-visual effect generation and mixing circuit 200. The radio transceiver 214 is further able to be part of an input that receives, over the wireless data link, audio-visual effects, including digitized sound signals, that are provided to other components of the audio-visual effect generation and mixing circuit 200, as is described below. The radio transceiver 214 of the exemplary embodiment is able to receive audio-visual effects from other wireless personal communications devices or from a base station 110.

The audio-visual effect generation and mixing circuit 200 of the exemplary embodiment includes a user input sensor 208 that generates an output in response to user motions that are monitored by the particular user input sensor. The user sensor 208 of the exemplary embodiment is able to include one or more sensors that monitor various movements or gestures made by a user of the wireless personal communications device 106. User sensors 208 incorporated in exemplary embodiments of the present invention include, for example, a touch sensor to detect a user's touching the sensor, a lateral touch motion sensor that detects a user's sliding a finger across the sensor, and an accelerometer that determines either a users movement of wireless personal communications device 106 itself or vibration of a cantilevered antenna, as is described below. A further user sensor 208 incorporated into the wireless personal communications device 106 of the exemplary embodiment includes a sound transducer in the form of a speaker that includes a feedback monitor to monitor acoustic waves emitted by the speaker that are reflected back to the speaker by a sound reflector, such as the user's hand. This allows a user to provide input by simply waiving a hand in front of the devices speaker. User sensor 208 is further able to include a sensor to accept any user input, including user sensors that detect an object's location or movement in proximity to the wireless personal communications device 106 as detected by, for example, processing datasets captured by an infrared transceiver or visual camera, as is discussed below.

The output of the one or more user input sensors 208 of the exemplary embodiment drives an audio-visual effects generator 210. The audio-visual effects generator 210 of the exemplary embodiment is able to generate digital sound information that includes actual audio signals, such as music, or definitions of sound effects that are to be applied to an audio signal, such as “wah-wah” effects, distortion, manipulation or generation of harmonic components contained in an audio signal, and any other audio effect. The audio-visual effects generator 210 further generates definitions of visual effects 224 that are displayed on visual display 222, such as lighting changes, graphical displays, kaleidoscope controls, and any other visual effects. The definition of visual effects 224 are further sent to a radio transmitter, discussed below, for transmission over a wireless data network, or sent to other visual display components, such as lights (not shown), within the wireless personal communications device 106 to locally display the desired visual effect.

The audio-visual effect generation and mixing circuit 200 of the exemplary embodiment further includes a sound source 204. Sound source 204 of the exemplary embodiment is able to include digital storage for music or other audio programming as well as an electrical input that accepts an electrical signal, in either analog or digital format, that contains audio signals such as music, voice, or any other audio signal. Further embodiments of the present invention incorporate wireless personal communications devices 106 that do not include a sound source 204.

The sound mixer 206 of the exemplary embodiment accepts an input from the sound source 204, from the audio-visual effects generator 210, and from the radio transceiver 214. The sound source 204 and the radio transceiver 214 of the exemplary embodiment produce digital data containing audio information. Sound source 204 is able to include an electrical interface to accept electrical signals from other devices, a musical generator that generates musical sounds, or any other type of sound source.

The sound mixer 206 of the exemplary embodiment mixes sound signals received from the sound source 204 and the radio receiver 214 to create sound information defining a sound input. The audio-visual effects generator 210 generates, for example, either additional sound signals or definitions of modifications to sound signals that produce specific sound effects. The sound mixer 206 combines the sound information defining the sound input with the generated audio-visual effects. This combining is performed by either one or both of modifying the sound information defining the sound input or by adding the generated additional sound signals to the sound input. The sound mixer 206 modifies sound signals by, for example, providing “Wah-Wah” distortion, generating or modifying harmonic signals, by providing chorus, octave, reverb, tremolo, fuzz, equalization, and by applying any other sound effects to the sound information defining the sound input.

The sound mixer 206 then provides the composite audio signal, which includes any sound effects defined by the audio-visual effects generator 210, to a Digital-to-Analog (D/A) converter 212 for reproduction through a speaker 230. The sound mixer further provides this composite audio signal to the radio transceiver 214 for transmission over the wireless data link to either a base station 110 or to other wireless personal communications devices 106.

The audio-visual effects generator 210 accepts definitions of visual effects received by the radio transceiver 214 over a wireless data link. The audio visual effects generator 210 may add to or modify these visual effects to create a visual effect output 224. The visual effects output 224 is provided to the radio transceiver 214 for transmission to either other wireless personal communications devices 106 or to a base station 110. The visual effects output 224 is similarly provided to a visual display 222 that displays the visual effects 224 in a suitable manner.

FIG. 3 illustrates a front-and-side view 300 of an exemplary monolithic wireless personal communications device 350 according to an exemplary embodiment of the present invention. The exemplary monolithic wireless personal communications device 350 is housed in a hand held housing 302. This exemplary hand held housing is holdable in a single hand. The exemplary monolithic wireless personal communications device 350 of the exemplary embodiment further includes a completely functional cellular telephone component that is able to support communicating over a commercial cellular communications system. The hand held housing 302 of the exemplary embodiment includes a conventional cellular keypad 308, an alpha-numeric and graphical display 314, a microphone 310 and an earpiece 312. The alpha-numeric and graphical display 314 is suitable for displaying visual effects as generated by the various components of the exemplary embodiment of the present invention. The exemplary monolithic wireless personal communications device 350 includes a cantilevered antenna 304 mounted or coupled to the hand held housing 302. An electrical audio output jack 316 is mounted on the side of the hand held housing 302 to provide an electrical stereo audio output signal in the exemplary embodiment that is able to drive, for example, a headset, an amplifier, an external audio system, and the like.

The exemplary monolithic wireless personal communications device 350 includes a touch sensor 306 that is a user input motion sensor in this exemplary embodiment. Touch sensor 306 is an elongated rectangle that detects a user's tap of the touch sensor with, for example, the user's finger. The touch sensor 306 further determines the tap strength, which is the force with which the user taps the touch sensor 306. The touch sensor 306 also determines a location within the touch sensor 306 of a user's touch of the touch sensor 306. The touch sensor 306 further acts as a lateral touch motion sensor that determines a speed and a length of lateral touch motion caused by, for example, a user sliding a finger across the touch sensor 306. In the exemplary embodiment, different audio-visual effects are generated based upon determined tap strengths, touch locations, lateral touch motions, and other determinations made by touch sensor 306.

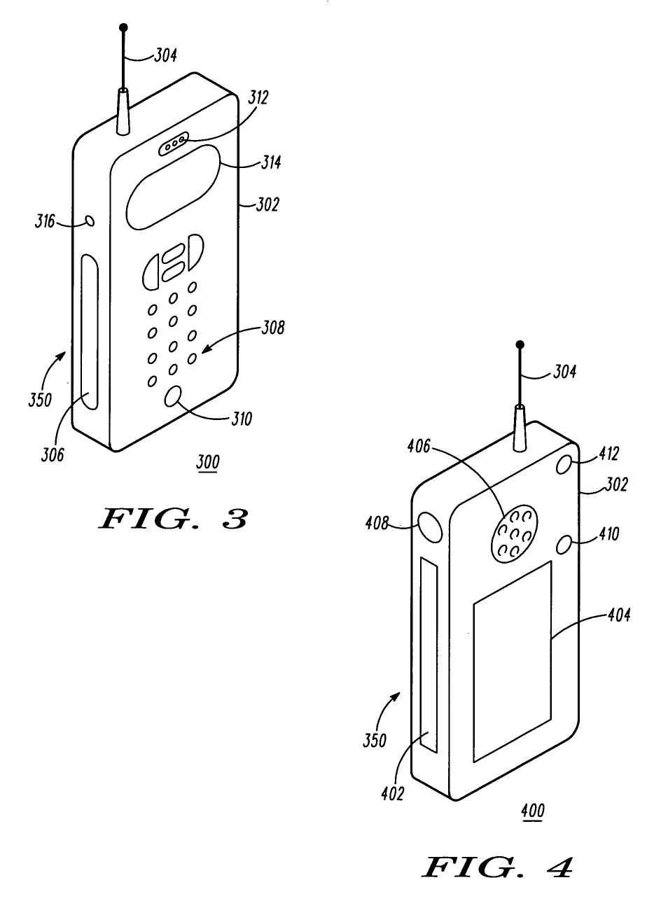

FIG. 4 illustrates a rear-and-side view 400 of an exemplary monolithic wireless personal communications device 350 according to an exemplary embodiment of the present invention. The rear-and-side view 400 of the exemplary monolithic wireless personal communications device 350 shows a palm rest pulse sensor 402 located on a side of the hand held case 302 that is opposite the touch sensor 306. The palm rest pulse sensor is able to monitor a user's pulse while holding the exemplary monolithic wireless personal communications device 350. The palm rest pulse sensor 402 of the exemplary embodiment is also able to monitor galvanic skin response for a user holding the exemplary monolithic wireless personal communications device 350. Alternative embodiments of the present invention utilize other pulse sensors, including separate sensors that are electrically connected to the exemplary monolithic wireless personal communications device 350. The rear-and-side view 400 of the exemplary monolithic wireless personal communications device 350 further shows an instrument input jack 408 mounted to the side of the hand held case 302. Instrument input jack 408 of the exemplary embodiment is a conventional one quarter inch jack that accepts audio signal inputs from a variety of electrical musical instruments, such as guitars, synthesizers, and the like.

The exemplary monolithic wireless personal communications device 350 further has a large touch sensor 404 mounted on the back of the hand held case 302. The large touch sensor 404 determines a tap strength, a touch location and lateral touch motion along the surface of the large touch sensor 404. The large touch sensor 404 of the exemplary embodiment is further able act as a fingerprint sensor that determines a fingerprint of a user's finger that is placed on the large touch sensor 404. Determining a user's fingerprint and altering the audio-visual effects based upon a user's finger print allows different users to generate different audio-visual effects and thereby create a personalized audio-visual style. The exemplary monolithic wireless personal communications device 350 further includes a loudspeaker 406 that is able to reproduce sound signals. The cantilevered antenna 304 is also illustrated.

An infrared transceiver 412 is further included in the monolithic wireless personal communications device 350 to perform wireless infrared communications with other electronic devices. The infrared receiver within the infrared transceiver 412 is further able to capture a dataset that can be processed to determine the amount of infrared energy that is emitted by the infrared transceiver 412 and that is reflected back to the infrared transceiver by an object located in front of the infrared transceiver 412. The infrared transceiver 412 is also able to determine an amount of infrared light that is emitted by an object located in front of the infrared transceiver 412. By processing a captured dataset to determine an amount of emitted or reflected infrared energy from an object, e.g., a piece of clothing that is placed in front of the infrared transceiver 412, the exemplary monolithic wireless personal communications device 350 is able to determine, for example, an estimate of the color of the object. The amount of reflected or emitted infrared energy is then able to be used as an input by the audio-visual effects generator 210 to control generation of different audio visual effects based upon that color. The infrared transceiver 412 of the exemplary embodiment is also able to process captured datasets to detect if an object is near the infrared transceiver 412 or if an object near the device moves in front of the infrared transceiver 412, such as hand motions or waving of other objects. The datasets captured by infrared transceiver 412 are able to include a single observation or a time series of observations to determine the dynamics of movement in the vicinity of the infrared transceiver 412. The distance or shape of an object that is determined to be within a dataset captured by the infrared transceiver 412 is able to control the generation of different audio-visual effects by the exemplary monolithic wireless personal communications device 350.

A camera 410 is further included in the exemplary monolithic wireless personal communications device 350 for use in a conventional manner to capture images for use by the user. The camera 410 of the exemplary embodiment is further able to capture datasets, which include a single image or a time series of images, to detect visual features in the field of view of camera 410. For example, camera 410 is able to determine a type of color or the relative size of an object in the field of view of camera 410 and the generated audio-visual effects are then able to be controlled based upon the type of colors detected in a captured image. As a further example of sound effects created by processing an image captured by camera 410, an image captured by camera 410 is able to include a photo of a person's body. The person's body is able to be determined by image processing techniques and a shape of the person's body, e.g., a ratio of height-to-width for the person's body, is able to be determined by processing the image data contained in the captured image dataset. A different sound effect is then able to be generated based upon the person's height-to-width ratio. A more specific example includes generating a low volume bass sound upon detecting a short, heavy set person, while detecting a tall slender person results in generating a high volume tenor sound.

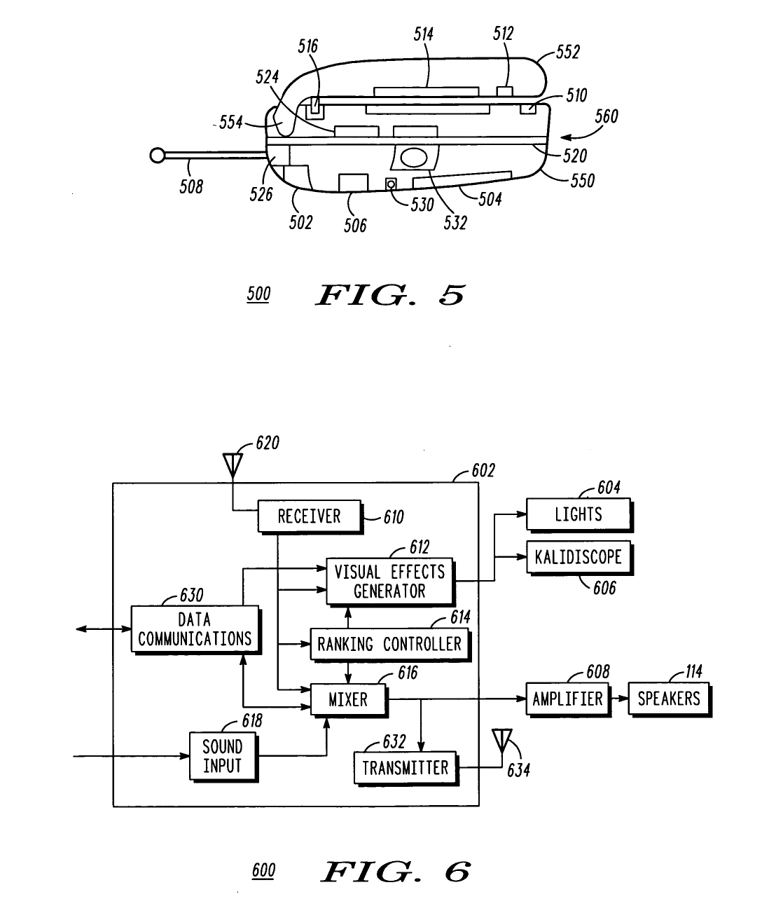

FIG. 5 illustrates a cut-away profile 500 of an exemplary flip-type cellular phone 560, according to an exemplary embodiment of the present invention. The flip type cellular phone 560 similarly has a capability to perform to support communicating over a commercial cellular communications system. The exemplary flip-type cellular phone 560 is housed in a two part hand held housing that includes a base housing component 550 and a flip housing component 552. This two part housing is holdable by a single hand. The flip housing component 552 of the exemplary embodiment has an earpiece 512 and display 514 mounted to an inside surface. The flip housing component 552 is rotatably connected to the base housing component 550 by a hinge 554. A flip position switch 516 determines if the flip housing component 554 is in a closed position (as shown), or if the flip housing component 552 is rotated about hinge 554 to be in an other than closed position.

The base housing component 550 includes a large touch pad 504 that is similar to the large touch sensor 404 of the exemplary monolithic wireless personal communications device 350 discussed above. The base housing component 550 further includes a loudspeaker 506 to reproduce audio signals and a microphone 510 to pick up a user's voice when providing voice communications. The base housing component 550 further includes an audio output jack 530 that provides an electrical stereo audio output signal in the exemplary embodiment that is able to drive, for example, a headset, an amplifier, an external audio system, and the like. The base housing component 550 further includes an instrument input jack 532 that is mounted on the side thereof. Instrument input jack 532 of the exemplary embodiment is a conventional one quarter inch jack that accepts audio signal inputs from a variety of electrical musical instruments, such as guitars, synthesizers, and the like.

The base housing component 550 also includes an accelerometer 502 that determines movement of the exemplary housing of the flip-type cellular phone 560 by the user, such as when a user simulates strumming a guitar or tapping a drum by waving the exemplary flip-type cellular phone 560. Accelerometer 502 is able to detect movements of the flip-type cellular phone 560 that include, for example, shaking, tapping or waving of the device. Accelerometer 502 is further able to detect a user's heart-beat and determine the user's pulse rate therefrom.

The base housing component 550 contains an electronic circuit board 520 that includes digital circuits 522 and analog/RF circuits 524. The analog/RF circuits include a radio transceiver used to wirelessly communication digital data containing, for example, audio-visual effects. The base housing component 550 of the exemplary flip-type cellular phone 560 includes a cantilevered antenna 508 mounts to an antenna mount 526. The antenna mount 526 electrically connects the antenna to electronic circuit board 520 and mechanically connects the antenna mount 526 to the base housing component and accelerometer 502. The mechanical connection of the cantilevered antenna 508 to the accelerometer 502 allows the accelerometer to determine vibrations in the cantilevered antenna 508 that are caused by, for example, a user flicking the cantilevered antenna 508. The frequency of vibration, which will be higher than a frequency of a user's waving of the exemplary flip-type cellular phone 560, is used by the exemplary embodiment to differentiate movement caused by waving of the exemplary flip-type cellular phone 560 and vibration of the cantilevered antenna 508. Additionally, a sensor contained within cantilevered antenna 508 detects in-and-out movement of a telescoping antenna. This in-and-out movement of the telescoping antenna is additionally used to control generation of sound effects or altering the speed at which a recorded work or a recorded portion of a work is being played back through the system.

FIG. 6 illustrates a collaborative audio-visual effect base station apparatus block diagram 600, according to an exemplary embodiment of the present invention. The audio-visual effect base station block diagram illustrates circuits within the exemplary base station 110 discussed above. The audio-visual effect base station 600 has a data processor 602 that includes a receiver 610 to receive data from a wireless data communications link that links, for example, multiple wireless personal communications devices 106. Receiver 610, which is coupled to antenna 620 to receive wireless communications signals, receives wireless digital data signals from contributing wireless personal communications devices that provide contributed audio-visual effects including audio signals, audio effect definitions, visual effect definitions. Receiver 610 further receives, from other wireless personal communications devices, data that includes user feedback, such as votes by spectators 108 using wireless personal communications devices 106, used to determine and maintain respective ratings or rankings for each individual performer 104 that is using a contributing wireless personal communications device 106 that is generating contributed audio visual effects.

The exemplary embodiment of the present invention allows spectators 108 to vote for individual performers who are able to be designated performers, such as musicians 104, or other spectators 108. Votes for individual performers are transmitted by the wireless personal communications devices 106 and received by receiver 610. These votes are provided to the ranking controller 614 which accumulates these votes and determines which performers' contributions are to be used as the audio-visual presentation or how much weighting is to be given to contributions from the various performers. Further, spectators 108 may rate for various performers in different categories, such as musical type (e.g., reggae, jazz, rock, classical, etc.). The ranking controller 614 of the exemplary embodiment maintains a ratings database that stores rating information for each performer. The rating for a respective individual is adjusted, over time, based upon the ratings information received from the spectators 108. The ratings database maintained by the ranking controller stores either an overall rating or a rating for each of various genres. For example, a particular performer is able to have different ratings for rock, reggae, and classical styles. The spectators are able to send ratings information for a particular performer to reflect either an overall rating overall rating or a rating for a particular genre. In an example, an embodiment of the present invention may have performers playing for a particular period of time in a specified genre, referred to as the current genre, and the spectators 108 are able to send in votes for the performers in this current genre.

Visual effect definitions received over the wireless data link by receiver 610 are provided to the visual effects generator 612. These visual effect definitions are combined based upon performer selections or weighting determined by the ranking controller 614. The ranking controller 614 determines selections or weightings based upon, for example, ratings stored in a ratings database as derived from default ratings for each performer and rating information received from spectators 108. For example, the ranking controller 614 is able to determine the top five ranked performers with regards to visual effects, and only their contributions are combined to provide visual effects. The ranking controller 614 is also able to define a weighting for each performer's input so that the contribution of the highest ranked performer is fully used to direct visual effects, and the contributions of lesser ranked performers are attenuated when producing the overall visual effect output.

The visual effects generator 612 is also able to receive visual effect definitions from data communications 630. Data communications 630 is connected to a data communications circuit, such as the Internet, and links the collaborative audio-visual effect base station 600 with remote locations, such as other venues or individual performers who are physically remote from the collaborative audio-visual effect base station 600.

The visual effects generator 612 of the exemplary embodiment is able to control lights 604 that illuminate a venue in which the performance is given. The visual effects generator 612 of the exemplary embodiment further controls a kaleidoscope 606 to provide visual effects.

The digitized audio signals received by receiver 610 are provided to mixer 616. Mixer 616 also receives audio signals through a sound input 618 that is able to accept, for example, recorded or live music. Mixer 616 is further able to accept digital music data from a data communications 630. The mixer 616 of the exemplary embodiment performs as a contribution controller that accepts rating information from each wireless personal communications device 106 within a plurality of wireless personal communications devices. Mixer 616 produces an audio-visual output that is derived from a plurality of audio-visual effects based upon the rating information by combining the audio-visual inputs according to performer selections and weightings determined by the ranking controller 614. The mixing of audio signals is able to be performed by, for example, selecting the five (5) highest ranking performers, or by mixing the contributions of various performers with weightings determined by their ranking.

The composite audio signal produced by mixer 616 is delivered to a transmitter 632 for transmission through antenna 634 to the multiple wireless personal communications devices 106. This optional feature allows the audio to be reproduced at each user's device instead of requiring a large speaker system. The composite audio output of mixer 616 is also able to be provided to an amplifier 608 for reproduction through speakers 114. Visual effects generated by mixer 616 are also sent to the visual effects generator 612 to be processed for display.

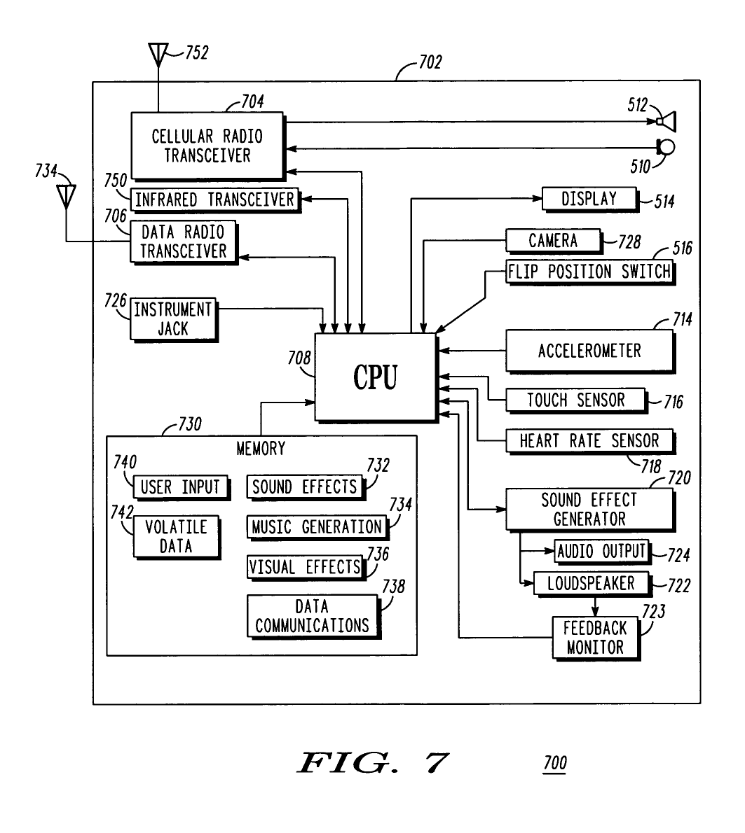

FIG. 7 illustrates a wireless personal communications device apparatus block diagram 700, according to an exemplary embodiment of the present invention. The wireless communications device apparatus block diagram 700 includes a wireless communications device 702 that is comparable to the exemplary flip-type cellular phone 560. The wireless communications device 702 is mechanically coupled to a cellular radio transceiver 704. The cellular radio transceiver 704 is a wireless personal communications circuit that provides voice and data communications over a commercial cellular communications system. The cellular radio transceiver 704 receives and transmits cellular radio signals through cellular antenna 752, processes and generates those cellular radio signals, and utilizes earpiece 512 and microphone 510 to provide audio output and input, respectively, to a user.

The wireless communications device 702 further has a data radio transceiver 706. The data radio transceiver 706 is a digital data wireless communications circuit that communicates with the wireless data communications circuit of the base station 110. The data radio transceiver 706 receives and transmits wireless data communications signals through data antenna 734 of the exemplary embodiment. As discussed above with respect to the base station 110, the data radio transceiver 706 of the wireless communications device 702 communicates using communications protocols conforming to the Bluetooth® standard and also includes data communications circuits that conform to data communications standards within the IEEE 802.11 series of standards. Further embodiments of the present invention are able to use any suitable type of communications, including cellular telephone related data communications standards such as, but not limited to, GPRS, EV-DO, and UMTS.

The wireless communications device 702 includes a central processing unit (CPU) 708 that performs control processing associated with the present invention as well as other processing associated with operation of the wireless communications device 702. The CPU 708 is connected to and monitors the status of the flip position switch 516 to determine if the flip housing component 552 is in an open or closed position, as well as to determine when a user opens or closes the flip housing component 552, which is an example of a motion performed in association with the housing. Some embodiments of the present invention generate an audio-visual effect, such as a drum noise, arbitrary noise or visual effect, in response to a user's opening and closing a flip housing component 552 of a flip type cellular phone 560.

CPU 708 of the exemplary embodiment is further connected to and monitors an accelerometer 714, touch sensor 716 and heart rate sensor 718. These sensors are used to provide inputs to the processing to determine the type of audio-visual effects are to be produced by the wireless communications device 702. CPU 708 further drives a sound effect generator 720 to produce sound effects based upon user inputs. The CPU 708 provides audio signals received by the data radio transceiver over the wireless data link to the sound effect generator 720. The sound effect generator 720 then modifies those audio signals according to sound effect definitions determined based upon user inputs, such as device waving determined by accelerometer 714, touching determined by touch sensor 716 and the user's heart rate determined by heart rate sensor 718.

The sound effect generator 720 is able to drive loudspeaker 722 to reproduce audio signals or provide the modified audio signal to CPU 708 for transmission by the data radio transceiver 706 to either another wireless communications device 702 or base station 110. The sound effect generator 720 further drives audio output jack 724 to provide an electrical output signal to drive, for example, headsets, external amplifiers or sound systems, and the like. A feedback monitor 723 receives reflected audio signals returned to the loudspeaker 722, as described below, to provide a user input that is provided to CPU 708.

CPU 708 of the exemplary embodiment is used to determine and create visual effects based upon user inputs. Visual effect definitions are able to be reproduced on display 514 or transmitted to a remote system, such as another wireless communications device 702 or base station 110, over the data radio transceiver 706.

CPU 708 is connected to a memory 730 that is used to store volatile and non-volatile data. Volatile data 742 stores transient data used by processing performed by CPU 708. Memory 730 of the exemplary embodiment stores machine readable program products that include computer programs executed by CPU 708 to implement the methods performed by the exemplary embodiment of the present invention. The machine readable programs in the exemplary embodiment are stored in non-volatile memory, although further embodiments of the present invention are able to divide data stored in memory 730 into volatile and non-volatile memory in any suitable manner.

Memory 730 includes a user input program 740 that controls processing associated with reading user inputs from the various user input devices of the exemplary embodiment. CPU 708 processes data received from, for example, the flip position switch 516, accelerometer 714, touch sensor 716, heart rate sensor 718 and feedback monitor 723. The raw data received from these sensors is processed according to instructions stored in the user input program 740 in order to determine the provided user input motion.

Memory 730 includes a sound effects program 732 that determines sound effects to generate in response to determined user input motions. User inputs used to control and/or adjust sound effects include movement of the wireless personal communications device 106 as determined by accelerometer 714, tapping or touching of touch sensor 716, the user's heart rate as determined by heart rate sensor 718, a user's galvanic skin response determined by touch sensor 716, a user's fingerprint detected by touch sensor 716, movement of a flip housing component 522 to operate the flip position switch 516, hand waving in front of loudspeaker 722 as determined by feedback monitor 723, or any other input accepted by the wireless personal communications device 106. Sound effect determined by CPU 708 based upon user inputs include “wah-wah” effects, harmonic distortions and any other modification of audio signals as desired. Different user input notions are able to be used to trigger different sound effects, such as hard taps of touch sensor 716 create one effect and soft taps create another effect. Sound effects can be personalized to individual users by detecting a user's fingerprint using touch sensor 716, such as a large touch sensor 404, and responding to various inputs differently for each detected fingerprint.

The CPU 708, under control of the sound effects program, provides sound information received by the data radio transceiver 706 to the sound effect generator 720 along with sound effect definitions or commands to control the operation of the sound effect generator in modifying the received sound information according to the determined sound effects. CPU 708 is further able to receive the modified sound information from the sound effect generator 720 and retransmit the modified sound information over a wireless data link through data radio transceiver 706. CPU 708 further accepts audio signals from an instrument jack 726. Instrument jack 726 of the exemplary embodiment is a conventional one quarter inch jack that accepts audio signal inputs from a variety of electrical musical instruments, such as guitars, synthesizers, and the like. The CPU 708 of the exemplary embodiment -includes suitable signal processing and conditioning circuits, such as analog-to-digital converters and filters, to allow receiving audio signals through the instrument jack 726.

Memory 730 includes a music generation program 734 that controls operation of CPU 708 in controlling the sound effect generator 720 in operating as a musical generator to generate musical sounds in response to user inputs. User inputs used to generate musical sounds include movements, such as shaking, tapping or waving, of the wireless personal communications device 106 as determined by accelerometer 714; a user's heart-beat rate as determined by vibrations measured by accelerometer 714; a color, size, distance, or movement of a nearby object as determined by either an infrared transceiver 750 or camera 728; a tapping, rubbing, or touching of touch sensor 716; a movement of a flip housing component 522 to operate the flip position switch 516; hand waving in front of loudspeaker 722 as determined by feedback monitor 723; or any other input accepted by the wireless personal communications device 106. The user is able to configure the wireless personal communications device 106 of the exemplary embodiment to produce different musical sound for different input sensors, or for different types of inputs to the different sensors. For example, a hard tap of touch sensor 716 may create a bass drum sound, a soft tap a snare drum sound and a stroking motion creates a guitar sound. These sounds are created by the sound effect generator 720 in the exemplary embodiment and are reproduced through loudspeaker 722 or communicated over a wireless data link via data radio transceiver 706.

Visual effects program 736 contained within memory 730 controls creation of visual effects, such as light flashing, kaleidoscope operations, and the like, in response to user inputs. User inputs that control visual effects are similar to those described above for audio effects. In a manner similar to audio effects and music generation, different user input motions are able to be assigned to different visual effects. The visual effects are communicated over a wireless data link via data radio transceiver 706 in the exemplary embodiment and are also able to be displayed by the wireless communications device 702, such as on display 514.

Wireless data communications, either over data radio transceiver 706 or over a cellular data like through cellular radio transceiver 704, is controlled by a data communications program 738 contained within memory 730.

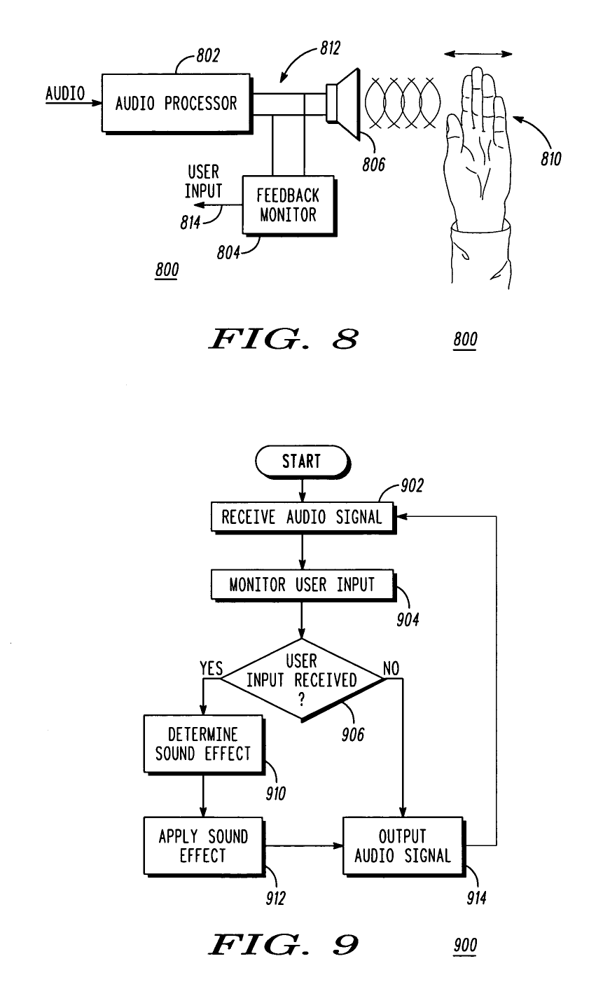

FIG. 8 illustrates a hand waving monitor apparatus 800 as incorporated into the exemplary embodiment of the present invention. The hand waving monitor circuit 800 is used to detect a motion of the user's hand in association with the housing as performed by the user of a wireless personal communications device 106. An audio processor 802 receives audio to be reproduced by a sound transducer such as loudspeaker 806. Audio processor 802 drives loudspeaker 806 with signals on speaker signal 812 and reproduces the audio signal. The audio signal in this example impacts a user's hand 810, which is placed in proximity to the loudspeaker 806, and is reflected back to loudspeaker 806. Loudspeaker 806 acts as a microphone and detects this reflected audio signal. The reflected audio signal creates an electrical disturbance on speaker signal 812 which is detected by an audio reflection monitor, which is the feedback monitor 804 of the exemplary embodiment, that is communicatively coupled to the sound transducer or loudspeaker 806. Movement of the user's hand 810, which is a sound reflecting surface, is detected by determining the dynamic characteristics of the feedback determined by feedback monitor 804. The feedback monitor 804 provides a conditioned output that reflects the user input 814 in order to control, for example, the audio-visual effect generator 210. The entire hand waving monitor apparatus 800 of this exemplary embodiment acts as a user input sensor 208.

FIG. 9 illustrates a sound effect generation processing flow 900 in accordance with an exemplary embodiment of the present invention. The sound effect generation processing flow 900 begins by receiving, at step 902, an audio signal. The audio signal in the exemplary embodiment is received, for example, over a wireless data link or by an electrically connected musical instrument or other audio source such as an audio storage, microphone, and the like. An audio signal is further able to be received through an instrument jack 726 from, for example, an instrument such as an electric guitar, synthesizer, and the like. The processing continues by monitoring, at step 904, for a user input from one or more user input sensors. The processing next determines, at step 906, if a user input has been received. If a user input has been received, the processing determines, at step 910, the sound effect to generate based upon the user input. Sound effects generated by the exemplary embodiment include modification of audio signals and/or creation of audio signals such as music or other sounds. The processing next applies, as step 912, the sound effect. Applying the sound effect includes modifying an audio signal or adding a generated audio signal into another audio signal that has been received. After the sound effect is applied, or if no user input was received, the processing outputs, at step 914, the audio signal. The audio signal in the exemplary embodiment is output to either a loudspeaker or transmitted over a wireless data link. The processing then returns to receiving, at step 902, the audio signal.

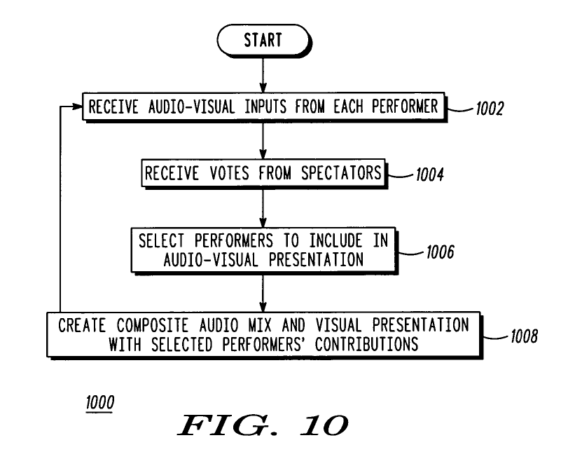

FIG. 10 illustrates a collaborative audio-visual effects creation system processing flow 1000 in accordance with an exemplary embodiment of the present invention. The collaborative audio-visual effects creation system processing flow 1000 begins by receiving, at step 1002, audio-visual inputs from each performer, such as musicians 104 or spectators 108. The processing next receives, at step 1004, votes from spectators for each musician or for musicians and selected spectators who are also selected to participate. The processing then selects, at step 1006, from which performers audio-visual contributions will be used to create a composite audio-visual presentation. This selection in the exemplary embodiment is able to be performed based on the votes received from spectators at step 1004. The processing is also able to select performers from whom contributions are used based upon, for example, random selection, cycling through all performers and optionally all spectators, or any other algorithm. Contributions from various selected performers are also able to be weighted based upon votes or any other criteria. The processing then creates, at step 1008, a composite audio mix and visual presentation with the selected performer's contributions. The processing then returns to receiving, at step 1002, audio-visual inputs from each performer.

The present invention can be realized in hardware, software, or a combination of hardware and software. A system according to an exemplary embodiment of the present invention can be realized in a centralized fashion in one computer system, or in a distributed fashion where different elements are spread across several interconnected computer systems. Any kind of computer system—or other apparatus adapted for carrying out the methods described herein—is suited. A typical combination of hardware and software could be a general purpose computer system with a computer program that, when being loaded and executed, controls the computer system such that it carries out the methods described herein.

The present invention can also be embedded in a computer program product, which comprises all the features enabling the implementation of the methods described herein, and which—when loaded in a computer system—is able to carry out these methods. Computer program means or computer program in the present context mean any expression, in any language, code or notation, of a set of instructions intended to cause a system having an information processing capability to perform a particular function either directly or after either or both of the following a) conversion to another language, code or, notation; and b) reproduction in a different material form.

Each computer system may include, inter alia, one or more computers and at least one computer readable medium that allows the computer to read data, instructions, messages or message packets, and other computer readable information. The computer readable medium may include non-volatile memory, such as ROM, Flash memory, Disk drive memory, CD-ROM, SIM card, and other permanent storage. Additionally, a computer medium may include, for example, volatile storage such as RAM, buffers, cache memory, and network circuits. Furthermore, the computer readable medium may comprise computer readable information in a transitory state medium such as a network link and/or a network interface, including a wired network or a wireless network, that allow a computer to read such computer readable information.

The terms program, software application, and the like as used herein, are defined as a sequence of instructions designed for execution on a computer system. A program, computer program, or software application may include a subroutine, a function, a procedure, an object method, an object implementation, an executable application, an applet, a servlet, a source code, an object code, a shared library/dynamic load library and/or other sequence of instructions designed for execution on a computer system.

Reference throughout the specification to “one embodiment” means that a particular feature, structure, or characteristic described in connection with the embodiment is included in at least one embodiment of the present invention. Thus, the appearances of the phrases “in one embodiment” in various places throughout the specification are not necessarily all referring to the same embodiment. Furthermore, the particular features, structures, or characteristics may be combined in any suitable manner in one or more embodiments. Moreover these embodiments are only examples of the many advantageous uses of the innovative teachings herein. In general, statements made in the specification of the present application do not necessarily limit any of the various claimed inventions. Moreover, some statements may apply to some inventive features but not to others. In general, unless otherwise indicated, singular elements may be in the plural and visa versa with no loss of generality.

While the various embodiments of the invention have been illustrated and described, it will be clear that the invention is not so limited. Numerous modifications, changes, variations, substitutions and equivalents will occur to those skilled in the art without departing from the spirit and scope of the present invention as defined by the appended claims.

What is claimed is:

1. A wireless personal communications device, comprising:
a hand held housing;
a wireless personal communications circuit, mechanically coupled to the housing, for communicating over a commercial cellular communications system or a peer-to-peer data communications network;
a user input sensor, mechanically coupled to the housing, able to detect a user input;
a sound input, mechanically coupled to the housing, for accepting an input containing sound information; and
an audio-visual effect generator, communicatively coupled to the user input sensor, that combines an audio-visual effect to the sound information based upon user input detected by the user input sensor.

2. The wireless personal communications device of claim 1, wherein the audio-visual effect generator comprises a sound signal modifier that electrically modifies the sound information, wherein the audio-visual effect generator combines the audio-visual effect to the sound information by modifying the sound information.

3. The wireless personal communications device of claim 1, wherein the user input sensor comprises a heart rate monitor to determine a user's heart rate, and wherein the audio-visual effect is adjusted based upon the user's heart rate.

4. The wireless personal communications device of claim 1, further comprising a sound transducer, wherein the user input sensor comprises an audio reflection monitor, communicatively coupled to the sound transducer, so as to detect the user input by monitoring movement of a sound reflecting surface proximate to the sound transducer.

5. The wireless personal communications device of claim 1, wherein the user input sensor comprises an infrared transceiver or a camera, wherein the audio-visual effect generator generates different audio-visual effects based upon a color, a distance, or a shape of an object determined to be within a dataset captured by the infrared transceiver or the camera.

6. The wireless personal communications device of claim 1, wherein the audio-visual effect generator further comprises a musical generator that generates musical sound, wherein the audio-visual effect comprises the musical sound, and wherein the audio-visual effect generator combines the audio-visual effect to the sound information by mixing the audio-visual effect with the sound information.

7. The wireless personal communications device of claim 1, wherein the user input sensor comprises a fingerprint sensor, wherein the audio-visual effect generator generates different audio-visual effects based upon a fingerprint determined by the fingerprint sensor.

8. The wireless personal communications device of claim 1, further comprising:
a wireless data communications link that communicates the audio-visual effect with other wireless personal communications devices; and
a speaker communicatively coupled to the audio-visual effect generator that produces at least a portion of the audio-visual effect; and
wherein the sound input comprises the wireless data communications link, and the wireless data link receives the sound information from the other wireless personal communications devices, and
wherein the audio-visual effect generator comprises a sound signal modifier that electrically modifies the sound information based on the audio-visual effect, and wherein the audio-visual effect generator combines the audio-visual effect to the sound information by modifying the sound information or generating a musical sound that is mixed with the sound information.

9. The wireless personal communications device of claim 1, wherein the user input sensor comprises an accelerometer that determines a motion of the housing to detect the user input.

10. The wireless personal communications device of claim 9, further comprising a cantilevered antenna that electromagnetically couples radio communications signals, wherein the accelerometer is mechanically coupled to the cantilevered antenna so as to monitor mechanical vibrations of the cantilevered antenna to detect the user input.

11. The wireless personal communications device of claim 1, wherein the user input sensor comprises a touch sensor that determines a tap strength, and wherein the audio visual effect generator generates different audio-visual effects based upon the tap strength, and wherein the touch sensor determines a touch location of a touch on the touch sensor, and wherein the audio-visual effect generator generates different audio-visual effects based upon the touch location.

12. A collaborative audio-visual effect creation system, comprising:
a plurality of audio-visual effect generators generating a plurality of audio-visual effects, each respective audio-visual effect generator within the plurality of audio-visual effect generators generating a respective audio-visual effect within the plurality of audio-visual effects;
a multiple user wireless data communications system that wirelessly communicates data among a plurality of wireless personal communications devices; and
a contribution controller that accepts rating information from each wireless personal communications device within the plurality of wireless personal communications devices, the contribution controller producing an audio-visual output derived from a plurality of audio-visual effects based upon the rating information.

13. The wireless personal communications device of claim 12, wherein the plurality of wireless personal communications devices comprises at least one contributing wireless personal communications device that each comprises an audio-visual effect generator that generates a contributed audio-visual effect, the contributed audio-visual effect comprising generating a musical sound, changing a lighting level, or modifying a kaleidoscope presentation.

14. The collaborative audio-visual effect creation system of claim 13, wherein the contributing wireless personal communications devices comprises:
a hand held housing;
a wireless personal communications circuit, mechanically coupled to the housing, for communicating over a commercial cellular communications system; and
a user input motion sensor able to detect at least one motion performed by a user in association with the housing; and
wherein the audio-visual effect generator is communicatively coupled to the user input motion sensor and generates the contributed audio-visual effect based upon motion detected by the user input motion sensor.

15. The collaborative audio-visual effect creation system of claim 12, wherein each contributing wireless personal communications device is operated by a respective user, wherein the contribution controller comprises a user ranking controller that maintains respective rankings for each user associated with each of the contributing wireless personal communications devices, and wherein the contribution controller produces the audio-visual output based upon the respective rankings.

16. The collaborative audio-visual effect creation system of claim 15, wherein the respective rankings are based upon a plurality of votes provided by users of the plurality of wireless personal communications devices.

17. A machine implemented method for generating an audio-visual presentation, the method comprising:
detecting at least one input from a user to a wireless communications device housing;
generating a first audio-visual effect based upon the at least one detected input;
receiving, over a wireless data link, a plurality of remotely generated audio-visual effects;
accepting rating information from a plurality of wireless personal communications devices; and
producing, based upon the rating information, an audio-visual output derived from the first audio-visual effect and the plurality of remotely generated audio-visual effects.

18. The machine implemented method of claim 17, wherein a portion of the plurality of wireless personal communications devices are located in a remote venue, the method further comprising communicating the audio-visual output to the remote venue.

19. The machine implemented method of claim 17, further comprising transmitting at least a portion of the audio-visual output to the plurality of wireless personal communications devices.

20. The machine implemented method of claim 17, wherein the rating information comprises votes for each of a plurality of individuals who are each generating portions of the plurality of remotely generated audio-visual effects, and wherein the producing the audio-visual output comprises selecting a subset of the plurality of remotely generated audio-visual effects and combining the subset to produce the audio-visual output, wherein the subset is selected based upon the rating information.

21. The machine implemented method of claim 20, further comprising maintaining a ratings database to store a rating for at least one of the plurality of individuals, wherein the rating for a respective individual is adjusted based upon the received ratings information, and wherein the producing the audio-visual output comprises selects the subset of the plurality of remotely generated audio-visual effects based upon ratings stored in the ratings database.

22. The machine implemented method of claim 21, wherein the ratings database stores, for at least one individual, an overall rating or a rating for each of a plurality of genres, and wherein the rating information comprises ratings for at least one individual for the overall rating or for a current genre.
